(19) United States
(12) Patent Application Publication
(10) Pub. No.: US 2022/0125602 A1
(43) Pub. Date: Apr. 28, 2022

(54) THREE-DIMENSIONAL PLANNING OF INTERBODY INSERTION

(71) Applicant: Mazor Robotics Ltd., Caesarea (IL)

(72) Inventor: Ido Zucker, Tel Aviv (IL)

(21) Appl. No.: 17/476,166

(22) Filed: Sep. 15, 2021

Related U.S. Application Data
(60) Provisional application No. 63/106,047, filed on Oct. 27, 2020.

(51) Int. Cl. A61F 2/46; A61B 34/10; G16H 20/40

(52) U.S. Cl. CPC A61F 2/4611; A61B 34/10; G16H 20/40; A61B 2034/104; A61B 2034/107; A61B 2034/105; A61F 2002/4633

(57) ABSTRACT

Embodiments include systems and methods for determining the treatment option most likely to result in a favorable long-term outcome in a subject with spinal pain. Using forms of computer learning and artificial intelligence, databases are generated and mined for information matching a subject of interest. Once the appropriate treatment option has been selected for the subject, if a surgical procedure is indicated, further methods are employed to select the optimal type of operation to eliminate the source of the pain and stabilize the spine. After the type of surgical procedure and approach to the area of interest have been determined, methods are described for selecting the optimal interbody for insertion under the control of a robotic surgical system. Additional methods are used for planning the minimal amount of bone that must be removed to allow insertion of intervertebral hardware such as an interbody.

### CROSS REFERENCE TO RELATED APPLICATIONS

This application claims priority to U.S. Provisional Application No. 63/106,047, filed on Oct. 27, 2020, and entitled “3-Dimensional Planning of Interbody Insertion”, the entirety of which is hereby incorporated by reference.

### FIELD

The present technology is generally related to the field of robotic surgery, especially for use in planning the insertion of tools or hardware in spinal surgery.

### BACKGROUND

Degenerative disease of the spine is a major cause of disability in the aging population. Vertebral pathology such as degenerative disc disease, herniated disc, or abnormal motion of spinal segments relative to each other often results in debilitating pain, necessitating surgical intervention in 20-30% of cases in which conservative treatment fails. Selecting the appropriate intervention for a given patient can relieve a significant amount of preoperative pain and disability. Several options are available for operative therapy, depending on the indication and source of pain. These include laminectomy or laminotomy to relieve pain caused by either spinal stenosis or a disc that may have herniated and impinged on the spinal cord or a spinal neve; artificial intervertebral disc replacement (AIDR) allowing range of motion, to substitute a degenerated disc; or fusion of adjacent vertebrae with insertion of an interbody cage in place of a degenerated disc, stabilizing the spine in a rigid conformation at the fused vertebrae. Lumbar AIDR without fusion has the potential to retain spinal flexion and may be indicated for patients suffering from significant axial back pain and/or radicular (nerve root) pain, secondary to disc degeneration or herniation, who have failed non-operative treatment. Alternatively, abnormal motion of the spinal segments may cause pain that can be relieved by fusing the involved segments.

A variety of surgical approaches exist, and numerous prosthetic interbodies are available on the market for replacement of an intervertebral disc. This variety gives the surgeon, in an operation on any given patient, many options to choose from. Thus, selection of the correct procedure is of critical importance. Regardless of which surgical therapy is chosen, the operative procedures to reach the area of interest are similar: in all cases, the internal spinal column must be reached by passing from the skin through a variety of soft tissues that must be protected from damage, and to insert an interbody or artificial intervertebral disc (AID), some amount of bone or ligament must be removed.

Below are listed resources pertaining to planning the surgical approach to spinal fusion or artificial intervertebral disc replacement.
Artificial disc replacement in spine surgery. Othman Y A, Verma R, Qureshi S A. Annals of Translational Medicine 2019; 7(Suppl 5): S170.
Spine Secrets [E-Book], Devlin V J. Elsevier Health Sciences, 13 Oct. 2020. Accessed online content Jul. 8, 2020.
The vertebral endplate: disc degeneration, disc regeneration. Moore R J; Eur Spine J (2006) 15(Suppl 3): S333-337.
Indications for lumbar total disc replacement: selecting the right patient with the right indication for the right total disc. Büttner-Janz K, Guyer R D, Ohnmeiss D D. Intl J of Spinal Surgery. 2015, Vol. 8, Art. 12.
How to choose when implants of adjacent height both fit the disc space properly in single-level cervical artificial disc replacement. Rong X, Lou J, Huibo Li H, Meng Y, Liu H. Medicine (2017) 96:29(e6954).
https://spinenation.com/treatment/lumbar-artificial-disc-replacement/6-lumbar-artificial-discs

The disclosures of each of the publications mentioned in this section and in other sections of the specification, are hereby incorporated by reference, each in its entirety.

### SUMMARY

The techniques of various embodiments described in this disclosure generally relate to methods of planning robotically-controlled insertion of surgical instrumentation or hardware to be implanted, otherwise known as an implant.

Considering the case of operative correction of a spinal pathology, an initial step is to determine the type of operation required. While in some cases the decision is clear cut, for other patients, more than one type of procedure may be considered. Embodiments of the present disclosure are configured to access a large database of results from prior operations and to classify the patient being considered in accordance with cases having similar pathologies and characteristics. Outcomes for the patients in the database are compared using artificial intelligence algorithms in order to select the type of operation most likely to result in a positive outcome for the patient in question. Once the type of operation to be performed has been selected, itis important to determine which surgical approach is likely to provide the minimal level of risk, the fastest recovery time and the greatest chances for long term successful outcome. In some embodiments, that determination can consider five surgical approaches generally used in the field. In other embodiments, the number of surgical approaches considered could be more or less. Considering five generally-used approaches to spinal surgery that requires access to the intervertebral disc space, these approaches can be divided into two major categories: anterior, which access the spinal column through the soft tissues of the abdomen and reach directly to the intervertebral space; and posterior, which require dissection of the posterior paraspinal muscles and removal of vertebral lamina and/or spinal processes to access the intervertebral disc space. Implementations of the methods and systems disclosed in embodiments of this disclosure generally relate to planning access and bone removal from the posterior direction, while planning access from the anterior direction is disclosed in another of the Applicant's applications, entitled “Surgical Path Planning using Artificial Intelligence for Feature Detection”, having the same inventor as the present application, and having docket number A0005235US01.

Insertion of an interbody in a spine procedure to replace a degenerated or slipped intervertebral disc is generally performed when using a posterior approach, by removing the vertebral lamina and clearing a path through the fascia and connective tissue so that the interbody can be inserted. The amount of bone to be removed is dependent upon the size of the interbody, the bone morphology and disease, and the position of the vertebrae during insertion. Currently, this process is usually performed manually by the surgeon through experience and intraoperative calculation.

In exemplary implementations of methods and a system for a spinal fusion procedure, the amount of bone to be removed to enable insertion of the hardware, and the tool path and movement through the soft tissue from a posterior or posterior-lateral approach, are determined, at least partially with the assistance of the automated system. A processor-based system calculates the approach angle and the entry point of the tool to the region of interest of the spinal column to minimize disruption to intervening soft tissue, specifically to the nerve roots. Based on processed images, the system may identify landmarks or regions that are forbidden for entry or removal, into which tools or hardware are thus excluded from entering. However, these forbidden regions may move during the operation. The system therefore takes into consideration not only the final desired position of the interbody, but also any changes in anatomical geometry, especially bony geometry, that the implant and tools may cause during delivery, and plans a path for the robotically guided tool which avoids forbidden structures and minimizes the amount of bone removed.

There is thus provided in accordance with an exemplary implementation of the devices described in this disclosure, a system of planning surgical access to an intervertebral disc space of a subject for robotic insertion of prosthetic hardware, thereby to create a bone-hardware assembly, the system comprising:

at least one processor executing instructions stored on at least one non-transitory storage medium, to cause the at least one processor to:

i) analyze collected clinical data on the subject to detect conditions that may affect either spine strength or the expected useful lifetime of the bone-hardware assembly;

ii) use a path-finding algorithm to plan a path for the surgical access using a virtual representation of the prosthetic hardware superimposed on a three-dimensional preoperative image set of the region of the intervertebral disc space of the subject; and

iii) use any of the detected conditions and the planned path to determine a minimal amount of vertebral bone to be removed to allow (a) removal of the intervertebral disc and (b) robotic insertion of the prosthetic hardware along the planned path, such that an increase is achieved in at least one of the likelihood of a favorable clinical outcome or the expected lifetime of the bone-hardware assembly.

In various exemplary implementations of the system, specific factors may change, as specified herewithin below. The minimal amount of vertebral bone to be removed may be determined using a path-finding algorithm. The type of prosthetic hardware may comprise an artificial intervertebral disc or an interbody cage. The outcomes of previous surgical procedures may comprise information on conditions that may affect spine strength; conditions that may affect spine strength comprise at least one of quality, osteoporosis, advanced age, and prior history of vertebral fracture. Conditions that may affect the expected lifetime of the bone-hardware assembly comprise at least one of previous surgery, preoperative instability, poor bone quality, degenerative bone disease, spondylolisthesis, and kyphotic deformity. The collected clinical data may comprise at least some of age, gender, height, weight, BMI, z-score, disc height, smoking history, number and identity of affected vertebral levels, underlying health conditions, the source of the intervertebral disc or spinal pathology, and analysis of the vertebral column in positions of bending. The three-dimensional preoperative image set may be one of MRI, CT, or reconstructed two-dimensional X-ray images. The structures susceptible to damage may comprise any of nerves, the thecal sac, blood vessels, muscles, or lymphatic vessels.

A favorable surgical outcome may be defined by at least two of a) long-term survival of the bone-hardware assembly, b) resolution of the subject's pain or loss of function, and c) lack of secondary complications from the surgical procedure resulting in injury to structures susceptible to damage. Minimizing the risk of failure of the bone-hardware assembly may take into consideration calculation and optimization of spinal alignment parameters. Planning of the minimal amount of vertebral bone to be removed may take into account protection of the vertebral end plates and avoidance of structures susceptible to damage.

The system may further comprise a step of programming a surgical robotic system to perform robotic insertion of the interbody after providing surgical access to the intervertebral disc space. The processor may be further configured to determine how much force is necessary to safely insert the interbody between the vertebrae. The system may further comprise a surgical robot having a controller configured to receive input from the processor, such that the surgical robot carries out the planned surgical access.

The system may be configured such that the at least one processor further uses training and inference logic to at least one of:

i) analyze the collected clinical data on the subject;

(ii) use a path-finding algorithm to plan the path for the surgical access; or

(iii) use any of the detected conditions and the planned path to determine a minimal amount of vertebral bone to be removed,

such that a greater increase is achieved in at least one of the likelihood of a favorable clinical outcome, or the expected lifetime of the bone-hardware assembly.

The system may be configured such that the bone-hardware assembly comprises either an inserted prosthetic intervertebral disc and its adjacent vertebral bodies; or at least one interbody and the associated hardware needed for a spinal interbody fusion.

There is further provided, in accordance with an exemplary implementation of the devices described in this disclosure, a method of planning surgical access to an intervertebral disc space of a subject, for robotic insertion of prosthetic hardware, thereby to create a hardware-bone assembly, the method comprising:

a) analyzing collected clinical data on the subject to detect conditions that may affect either spine strength or the expected lifetime of a prosthetic hardware-bone assembly;

b) using virtual representations of the prosthetic hardware and a three-dimensional preoperative image set of the surgical region of the subject, applying a path-planning algorithm to enable insertion and positioning of the prosthetic hardware; and

c) determining the minimal amount of vertebral bone to be removed by a robotic system to allow (a) removal of the intervertebral disc and (b) insertion of the prosthetic hardware along the planned path, taking into account any of the detected conditions, such that an increase is achieved in at least one of the likelihood of a favorable clinical outcome or the expected lifetime of the prosthetic hardware-bone assembly.

In various exemplary implementations of the disclosed method, specific factors may change, as specified herewithin below. The minimal amount of vertebral bone to be removed may be determined using a path-finding algorithm. The type of prosthetic hardware may comprise an artificial intervertebral disc or an interbody cage. Conditions that may affect spine strength may comprise at least one of poor bone quality, osteoporosis, advanced age, and prior history of vertebral fracture, and conditions that may affect the prosthetic hardware-bone assembly comprise at least one of previous surgery, degenerative bone disease, spondylolisthesis, and kyphotic deformity.

The collected clinical data may comprise at least some of age, gender, height, weight, BMI, z-score, disc height, smoking, vertebral levels, underlying health conditions, the source of the subject's intervertebral disc or spinal pathology and analysis of the vertebral column in positions of bending. The three-dimensional preoperative image set may be one of MRI, CT, or reconstructed two-dimensional X-ray images. A favorable surgical outcome is defined by at least two of a) long-term survival of the prosthetic hardware-bone assembly, b) resolution of the subject's pain or loss of function, and c) lack of secondary complications from the surgical procedure.

Increasing the expected lifetime of the prosthetic hardware-bone assembly may take into consideration calculation and optimization of spinal alignment parameters. The processor may be further configured to determine how much force is necessary to safely insert the interbody between the vertebrae.

The method may further comprise a step of programming a surgical robotic system to perform robotic insertion of the interbody after providing surgical access to the intervertebral disc space. The structures susceptible to damage may comprise any of nerves, the thecal sac, blood vessels, muscles, or lymphatic vessels.

There is further provided, in accordance with an exemplary implementation of the devices described in this disclosure, a system for determining the suitability of a subject with spinal pain, for a surgical procedure to decompress or replace an intervertebral disc, the system comprising:

at least one processor executing instructions stored on at least one non-transitory storage medium, to cause the at least one processor to:
i) analyze a database of medical history information of a reference population comprising patients having previously undergone surgical procedures for spinal pain, to categorize outcomes of the surgical procedures according to clinical and demographic parameters;
ii) use the analyzed database to classify the subject based on the clinical and demographic parameters of the subject; and
iii) based on the classification of the subject, determine at least one of:
   a) the suitability of the subject for surgical treatment;
   b) the type of surgical procedure to perform on the subject; or
   c) the surgical approach to perform the surgical procedure;

wherein the determination results in optimization of the expected outcome of the surgical procedure on the subject.

In further exemplary implementations or modifications of the disclosed exemplary system for determining the suitability of a subject with spinal pain, for a surgical procedure to decompress or replace an intervertebral disc, categorizing outcomes of surgical procedures may comprise ranking the degree of spinal pain and the physical disability of patients preoperatively and post-operatively according to a numerical scale. The numerical scale may be either of the Neck Disability Index (NDI) or the Oswestry Disability Index (ODI). The determination of suitability for surgical treatment may be based on consideration of at least one of clinical and demographic factors, underlying bone disease, or preexisting conditions. The type of surgical procedure may comprise one of foraminotomy, laminectomy, laminotomy, artificial intervertebral disc replacement, or spinal fusion. The surgical approach to perform the surgical procedure may comprise one of anterior, oblique, lateral, transverse, or posterior approaches. The expected outcome of the surgical procedure may be defined by at least two of a) long-term survival of the bone-hardware construct, b) resolution of the subject's pain or loss of function, or c) reduced secondary complications from the surgical procedure. Classifying clinical and demographic parameters of the subject may comprise matching at least some of the subject's clinical presentation of spinal pain, concurrent medical conditions, and demographic data with the analyzed medical history information of the reference population showing outcomes of surgical procedures.

There is further provided, in accordance with an exemplary implementation of the devices described in this disclosure, a system for planning the selection of an artificial prosthesis to replace an intervertebral disc, comprising:

a) a memory configured to store a selected surgical procedure and a surgical approach for performing insertion of an artificial disc prosthesis on a subject,

b) a channel providing access to information on at least some of dimensions, shape, indicated surgical use, indicated vertebral levels, material composition, and success rate of available artificial disc prostheses, and

c) a controller accessing artificial intelligence algorithms, to i) analyze the information on the available artificial disc prostheses, and ii) select an artificial disc prosthesis for the subject, such that the long term outcome of the surgical procedure on the subject is optimized.

In further exemplary implementations or modifications of the disclosed exemplary system for planning the selection of an artificial prosthesis to replace an intervertebral disc, the artificial prosthesis may comprise one of an artificial intervertebral disc or an interbody. The surgical procedure to be performed may comprise one of an artificial intervertebral disc replacement or a spinal fusion. The surgical approach may comprise one of anterior, lateral, oblique, transverse or posterior. The at least one of the algorithms may take into account the optimal height and lordotic angle of the intervertebral disc to be replaced by the artificial prosthesis. Optimizing the expected outcome of the surgical procedure may be defined by at least two of a) long-term survival of the artificial prosthesis, b) resolution of the subject's disability, and c) lack of secondary complications from the surgical procedure.

The system for planning the selection of an artificial prosthesis may further comprise training data on outcomes of previous surgical procedures and surgical approaches using the available artificial disc prostheses. The training data may be used by the algorithms to predict at least one of a) long-term survival of the artificial prosthesis, b) resolution of the subject's disability, and c) lack of secondary complications from the surgical procedure.

There is further provided, in accordance with an exemplary implementation of the devices described in this disclosure, a method for planning the selection of an artificial prosthesis to replace an intervertebral disc, the method comprising:

a) selecting a surgical procedure and surgical approach for performing insertion of an artificial disc prosthesis on a subject;

b) inputting into a processor information comprising at least some of dimensions, shape, surgical indication, vertebral level, material composition, and success rate on available artificial disc prostheses; and

c) using the processor, selecting an artificial disc prosthesis that optimizes the possibility for a long term outcome of the surgical procedure on the subject, defined by at least two of a) long-term survival of the artificial prosthesis, b) resolution of the disability of the subject, or c) lack of secondary complications from the surgical procedure.

In further exemplary implementations or modifications of the disclosed exemplary method for planning the selection of an artificial prosthesis to replace an intervertebral disc, the artificial disc prosthesis to replace the intervertebral disc may comprise one of an artificial intervertebral disc, or an interbody for spinal fusion. The surgical procedure to be performed may comprise one of an artificial intervertebral disc replacement or a spinal fusion. The surgical approach may comprise one of anterior, lateral, oblique, transverse or posterior approach. The method may further take into account the optimal height and lordotic angle of the intervertebral disc to be replaced by the artificial prosthesis.

It is to be appreciated that any feature described herein can be claimed in combination with any other feature(s) as described herein, regardless of whether the features come from the same described embodiment.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below. Numerous additional features and advantages of the present invention will become apparent to those skilled in the art upon consideration of the embodiment descriptions, drawings, and claims provided hereinbelow.

The details of one or more aspects of the disclosure are set forth in the accompanying drawings and the description below. It is to be understood that the scope of the methods revealed in this disclosure have many more applications than is possible to describe in their entirety. One exemplary implementation is thus described in detail, with other possibilities suggested in brief. Additional features, objects, and advantages of the techniques will be apparent from the description and drawings, and from the claims.

### Definitions

Artificial intervertebral disc (AID): As opposed to an interbody (see below), an AID is an implant intended to replace the natural disc in a way that reproduces at least some of the normal range of motion provided by a natural intervertebral disc. Several models are currently available, and new models are under development and testing.

Interbody cage: A prosthetic spacer with a hollow space for a bone graft. An implant used to replace an intervertebral disc in a spinal fusion operation in combination with hardware such as fusion rods to immobilize the fused joint; also referred to as a spinal implant, an interbody, and an interbody prosthesis.

Spinal interbody fusion: A surgical operation to fuse two or more vertebrae in a procedure requiring the insertion of an interbody cage between the bodies of two vertebrae being fused, in combination with fusion rods to secure adjacent vertebrae.

Surgical approach: For accessing the intervertebral space for either artificial intervertebral disc replacement or interbody fusion, the surgeon must dissect a path to the internally located tissues. Five main surgical approaches have been developed for carrying out these procedures. These are anterior lumbar interbody fusion (ALIF), oblique lumbar interbody fusion (OLIF), lateral lumbar interbody fusion (LLIF), posterior lumbar interbody fusion (PLIF), and transforaminal lumbar interbody fusion (TLIF) approaches.

### BRIEF DESCRIPTION OF DRAWINGS

FIG. 1 is a flowchart illustrating an overview of an implementation of the disclosed methods;

FIG. 2 is a flowchart describing the steps in selection of a particular operation, surgical approach, and spinal hardware selection;

FIG. 3 is a flowchart showing the steps involved in an exemplary implementation of the bone removal process and interbody or AID insertion;

FIG. 4 is a flow diagram that illustrates presurgical planning to select the procedure likely to have the greatest benefit for the patient;

FIG. 5 is a flow diagram with a decision tree illustrating an exemplary method of selection of various types of AID and interbodies for insertion;

FIGS. 6A-6D illustrate schematically surgical bone removal and interbody insertion in an exemplary implementation of the disclosed methods; and

FIG. 7 is a block diagram/conceptual that illustrates the structural components that comprise the system used to carry out some implementations of the disclosed methods.

### DETAILED DESCRIPTION

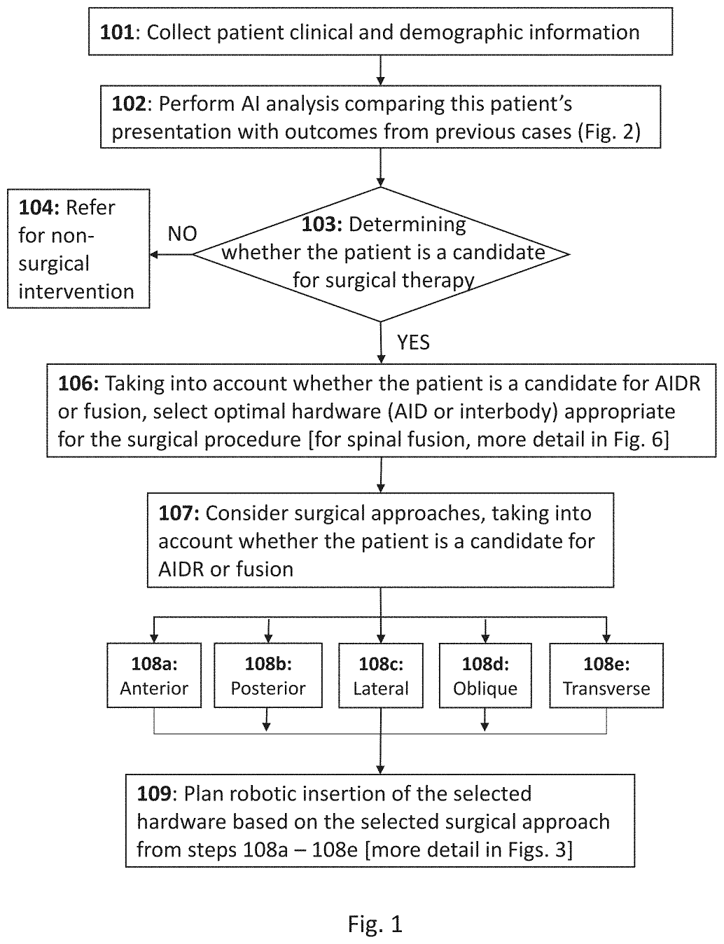

Reference is made to FIG. 1, showing an overview of an exemplary implementation of disclosed methods for evaluating and treating a subject presenting with chronic back pain, to determine whether a surgical procedure is indicated, and exemplary methods of deciding which surgical procedure to implement in the case that surgical treatment is indicated. While some embodiments of the present disclosure uses lumbar fusion or AIDR as an exemplary implementation of the disclosed methods, variations of the same procedures are applicable to disc procedures in other parts of the spine, such as the cervical spine.

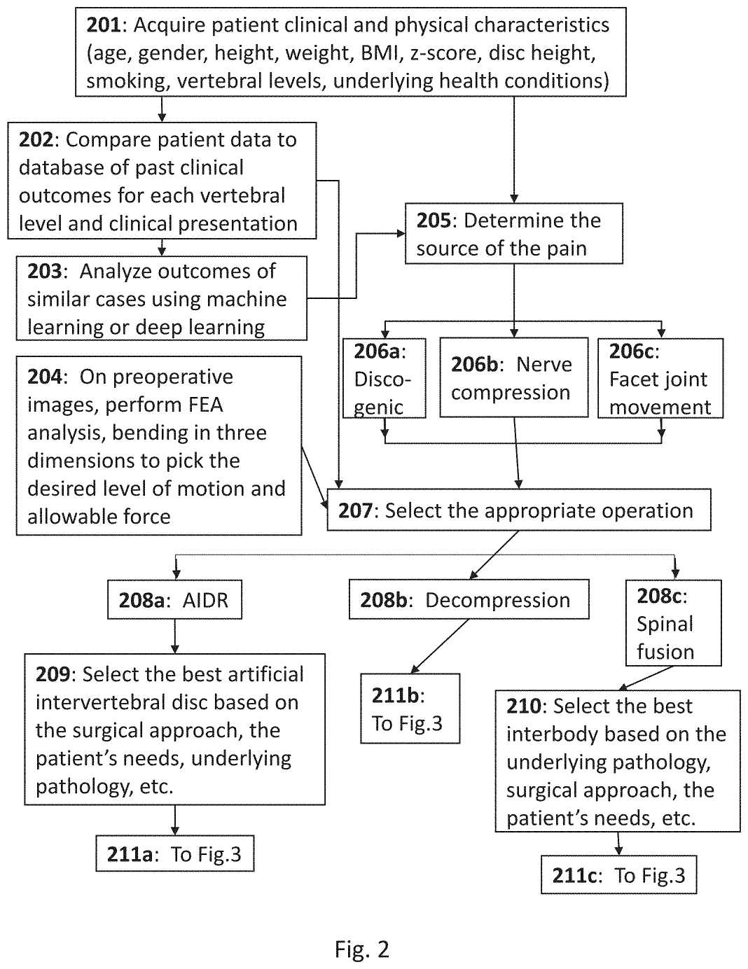

In step 101, clinical and demographic information is collected from the patient. These data comprise past medical history, imaging studies, physical findings, physical therapy recommendations, pain evaluation to map the points of pain, and any other relevant tests that may have been performed such as electromyography. In step 102, the data collected in step 101 are subjected to analysis using artificial intelligence algorithms such as machine learning, deep learning, neural networks, or other type of data analytics. This analysis is based on a database of prior cases with outcomes and long term follow-up. Further details of this process are described in more detail in FIG. 2. In step 103, based on the output of step 102, the system evaluates the suitability of the patient for surgical intervention. If the individual is deemed inappropriate for an invasive procedure, in step 104, he/she is referred for non-surgical intervention.

If the patient is a candidate for surgery, determination is made as to whether a lumbar fusion or AIDR would be likely to provide the best outcome for that patient. The decision depends on a number of factors, such as age, degree of disability, and the potential for significant recovery of range of motion in the vertebra(e) undergoing repair. For example, the ideal patient for AIDR is preferably less than 45 years old, with back pain severe enough to impact activities of daily living and/or work. The primary clinical indication for AIDR is symptomatic degenerative disc disease with or without radicular pain. Examples of cases in which AIDR may be indicated comprise: discogenic low back pain caused by osteochondrosis; sciatica associated with degenerative spondylolisthesis, a lack of significant psychological issues, and diagnostic studies confirming the disc as the pain generator. All major contraindications should preferably be absent. In other implementations of the disclosed methods, additional surgical procedures may be included in the options, such as laminotomy or laminectomy.

After the surgical operation has been decided, in step 106, the optimal implant is selected: if the operation is AIDR, a suitable Artificial Intervertebral Disc (AID) is selected; if the procedure is spinal fusion, an appropriate interbody is selected. This selection is also based on artificial intelligence analysis of the outcomes of prior cases. Most cases of AIDR are performed using an anterior approach, and most cases of spinal fusion are performed using one of the posterior approaches. Many configurations for AID are currently available, with additional types anticipated in the future. Various embodiments described herein have the ability to incorporate information about any new artificial intervertebral discs that may become clinically relevant in the future. Each type of AID is suitable for one or more surgical approaches to disc replacement. Typically, AID are inserted anteriorly, although posterior and other standard surgical approaches to the spine are also in use. The analysis incorporates resources that provide the full range of indications and contraindications for the selected device. If the selected operation is a lumbar fusion, a suitable interbody cage is chosen. For both AIDR and fusion, the choice will incorporate information on a number of factors, comprising the vertebral level of operation, the surgical approach, patient clinical and other data from step 101.

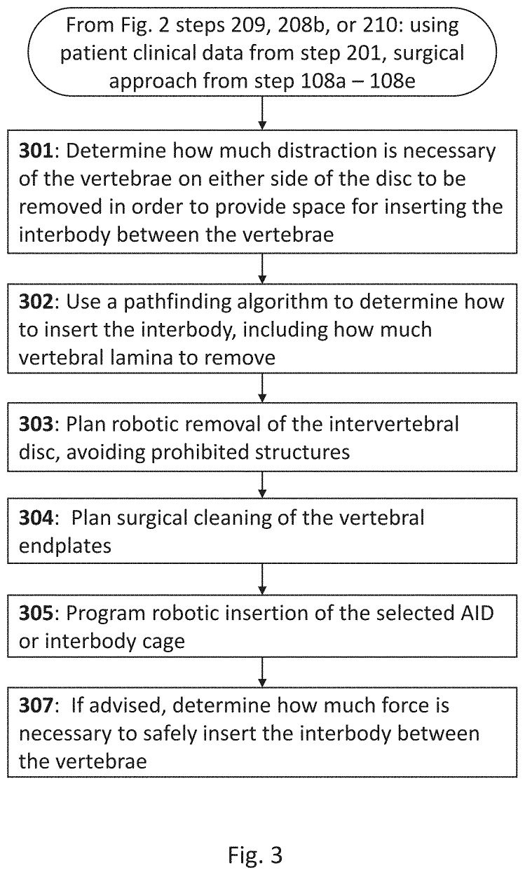

In step 107, the system evaluates which surgical approach would have the highest probability of providing the best outcome for this patient. This determination is made by data analytics evaluating the outcomes of past cases, wherein the subject's clinical history and characteristics are matched as closely as possible with prior patient history and characteristics. In an exemplary implementation of the method, the standard surgical approaches to the disc space are the same for either AIDR or fusion; as listed in steps 108a to 108e, they are anterior, posterior, lateral, oblique, and transverse. In step 109, the surgeon or the system plans the robotic insertion of the selected hardware based on the selected surgical approach of steps 108a-108e, as described in more detail in FIG. 3 and below.

Reference is now made to FIG. 2, showing an additional component of planning the surgical path required in some surgical approaches, and more detail about the initial steps in FIG. 1. It is to be noted that the options presented in the figure are exemplary method routines for some possible clinical scenarios, and the scope of the disclosure is in no way meant to be limited by these examples. In step 201, corresponding to step 101, patient clinical history and physical characteristics are acquired. History and characteristics are not limited to, but comprise at least some of, age, gender, height, weight, BMI, z-score (or DEXA T-score), disc height, smoking history, psychosocial and psychological history, vertebral levels affected by disease, underlying health conditions, and previous history of surgical and non-surgical intervention. In step 202, patient data is compared with clinical data in at least one database comprising information regarding past clinical outcomes for each vertebral level and clinical presentation. This database, or another, may also comprise detailed information culled from the scientific and medical literature on the findings of experimental models, patient outcomes, and other relevant findings. In step 203, the data from step 202 is analyzed using machine learning, deep learning, or other form of data analytics applying artificial intelligence to learn the source of the pain based on previously treated cases. In optional step 204, finite element analysis (FEA) may be performed under conditions of bending in various directions, to select the desired level of motion and allowable force. In step 205, based on the information provided by steps 201 to 203, the source of the pain is determined. This step may be performed by either the system, or is based on the clinical examination performed by a member of the medical staff, or by artificial intelligence analysis of the presented data. In step 206a to 206c, the source of pain is categorized as most likely to be discogenic, i.e., due to disintegration of the disc itself (206a), from nerve or cord compression due to a herniation or stenosis (206b), or from abnormal movement of the facet joints (206c). In step 207, approximately corresponding to steps 106 to 108, and using as input the output of steps 203 and 206a-206c, the system selects the most appropriate operation for the individual subject, choosing among AIDR (208a), laminectomy or laminotomy (208b), or spinal fusion (208c). Each option is generally suitable for the corresponding source of pain directly above it in FIG. 2, steps 206a-c, although other cross-barrier options may be selected as most appropriate.

It is noted that the disclosed methods are not limited to the procedures and steps disclosed in typical implementations of the methods described here, but are generally applicably to a variety of conditions, for example, neck pain and surgical treatment of the cervical vertebrae.

If the surgical procedure chosen is AIDR (208a) or spinal fusion (208c), the system determines which surgical approach to use, as shown in steps 108a-e, and as described in more detail in the above mentioned co-pending Patent Application entitled “Surgical Path Panning using Artificial Intelligence for Feature Detection”, having a common inventor with the present application. In some implementations of the disclosed methods, the system further evaluates options such as selecting between a laminectomy or a laminotomy. Once the system selects the surgical approach determined to have the highest likelihood of success for the subject as described in step 107 hereabove, the method proceeds to either step 209, in the case of an AIDR, or step 210, in the case of a spinal fusion. In step 209, the most suitable AID is selected based on the surgical approach, the patient's needs, the underlying pathology, and other factors.

In step 210, further planning of a spinal fusion procedure is carried out. A spinal fusion requires the implantation of hardware. Therefore, after or in combination with selecting the surgical approach, the system may make use of methods disclosed in WO2018/131044 Dynamic Motion Global Balance, WO2016/088130 Shaper for Vertebral Fixation Rods, WO2017/064719 Global Spinal Alignment Method, WO2018/131044 Dynamic Motion Global Balance, and other disclosures having a common assignee as the present application. These other methods are part of the surgical planning procedure and are used to assist the surgeon in deciding which and how many vertebral levels to instrument, how to bend the hardware, and other aspects of the planned procedure. The best one or more interbodies is selected, based on the patient's underlying pathology, vertebral levels affected by disease and planned to be fused, and surgical approach, further taking into account the anticipated useful lifetime of the bone-hardware assembly relative to the expected life-span of the patient. Depending on which surgical procedure is selected, following any of steps 209, 208b, and 210, the method proceeds to FIG. 3.

Reference is now made to FIG. 3, showing steps involved in planning the robotic execution of the surgical procedure. In step 301, if the chosen approach is PLIF or another that requires surgical access to the intervertebral space from the posterior direction in order to allow insertion of the interbody, the system must determine how much of the vertebral lamina is necessary and safe to remove. Removing the lamina and underlying ligament gives more room for decompression and for insertion of the prosthetic interbody; however, depending on the amount of tissue removed and whether the spine has been weakened by degenerative changes or previous surgery, the strength of the spine may be compromised by disrupting the bone and ligament in this area. Thus, to ensure an optimal outcome within the constraints of an individual patient's anatomy, an assessment of these two possibly opposing factors is needed: for providing optimal access to the intervertebral space, removing more bone is better, whereas for preserving the strength of the vertebral column, less bone removal is better.

The system determines how much vertebral lamina to remove using a pathfinding algorithm and incorporating:

a) patient clinical data from step 201,

b) the surgical approach from step 109, and

c) the selected AID or interbody cage from step 210 or 211.

In step 303, the system plans robotic removal of the nucleus pulposus of the intervertebral disc, avoiding prohibited structures. Execution of the actual procedure may be assisted by the systems and methods disclosed in U.S. 62/952,958 for “Endoscopic Ultrasound Robotic Guidance” or in WO 2010/064234 for “Robot Guided Oblique Spinal Stabilization”, both co-assigned to the present applicant. In step 304, the system plans surgical cleaning of the vertebral endplates, taking care not to damage the bony surfaces. This can be a crucial step, as the endplates are only 1-2 mm in thickness, and both critical for vertebral preservation and easily damaged. Furthermore, a complete disc resection is necessary to maximize the fusion surface area. In step 305, the system plans robotic insertion of the selected AID, in the case of an AIDR, or at least one interbody, in the case of a spinal fusion. In step 306, further determination is made of how much distraction is necessary to provide space for inserting the interbody between the vertebrae. In step 307, the system determines how much force is necessary to safely insert the interbody or the AID between the vertebrae. In the case of robotic execution of the procedure, the amount of force to be applied must be carefully evaluated. The resistance normally encountered and used as feedback during human insertion of the hardware may be automatically sensed by a sensor configured to sense the power applied to the motors involved in the hardware insertion. Alternatively, force sensors may be incorporated into the robotic arm or into the surgical tool used for inserting the hardware, for the sake of providing feedback and preventing an excess level of force being used on the tissue.

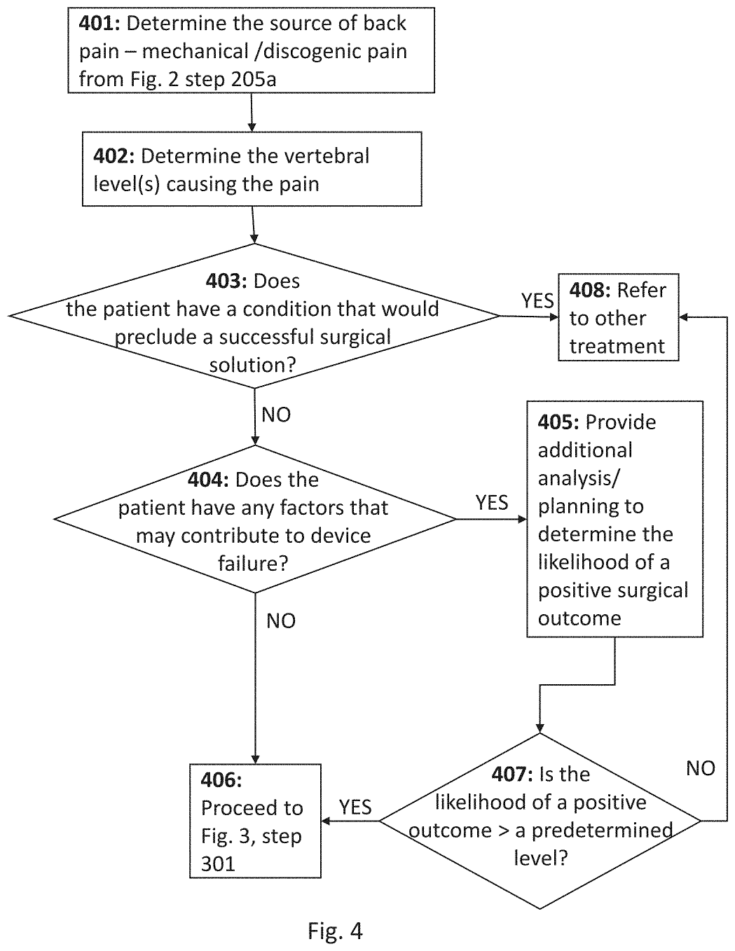

Reference is now made to FIG. 4, a flowchart outlining basic steps taken in one implementation of the disclosed methods to evaluate the source of low back pain and to determine the approach to treatment most likely to succeed. A similar set of rules would be followed for neck pain caused by cervical spinal disease, with specific adaptations of the parameters evaluated based on the level or region of the spine affected by disease. Each main step comprises a number of substeps to evaluate additional details.

Step 401 follows from FIG. 2 step 205a, i.e., the method begins once the etiology of the low back pain in the subject under evaluation has been identified as mechanical or discogenic. This determination is made by the machine learning or deep learning algorithms that evaluate the patient's data in view of previous cases, from a large database of information. The database information may be derived from previous case documentation, from anonymous data from health insurance companies, data culled from the published medical literature, or other sources of health databanks.

In step 402, a determination is made of the vertebral level affected, based on results of at least one of physical exam and imaging studies. More than one level may be affected, e.g., both L3 and L4 discs may be diseased and need repair.

In step 403, the system evaluates whether the patient has a disorder that reduces the strength of the vertebral body, or a degree of spondylolisthesis, or anterior-posterior shifting of one vertebral bodies relative to another, greater than Grade 1. The condition may be osteoporosis, disc space infection or systemic infection, unhealed spinal fracture at the level of the disease, spinal tumor, vertebral body cyst, or other disease. If so, in step 408 the patient may be referred to a non-surgical intervention, using a conservative approach such as physical therapy. If not, the method proceeds to step 404, in which an evaluation is made to determine whether the patient has any of the following factors that could contribute to the failure of a prosthetic AID or interbody cage, such as preoperative instability, poor bone quality, or kyphotic deformity. If not, the method proceeds to step 406, returning to FIG. 3 step 301 to plan the surgical procedure, if the approach is from the posterior aspect of the patient. If the approach is an anterior approach, the system directs the process to the methods disclosed in the above-mentioned co-pending US Patent Application “Surgical Path Planning using Artificial Intelligence for Feature Detection”, having a common assignee with the present application.

If in step 404, the system determines that the patient has factors that may contribute to failure of an implanted AID, the method proceeds to step 405, in which additional analysis or planning is performed to determine the likelihood of a positive surgical outcome. This analysis comprises further comparison of the patient's data with the database of clinical and surgical outcomes with a finer level of detail and focus on long-term as well as short-term results in the general population represented in the database. In step 407, the system determines whether the likelihood of a positive outcome in this case is above a predetermined percentage, for example, more than 70%. If not, the process proceeds to step 408, in which the patient is referred for other, i.e., non-surgical, treatment. If so, the system proceeds to step 406, for surgical procedure planning as described above. Examples of factors that may be considered at this phase of the planning process and which may affect the success of a surgical procedure are T score (reflecting bone density), serum vitamin D levels, smoking history, BMI, spinal deformity, and age.

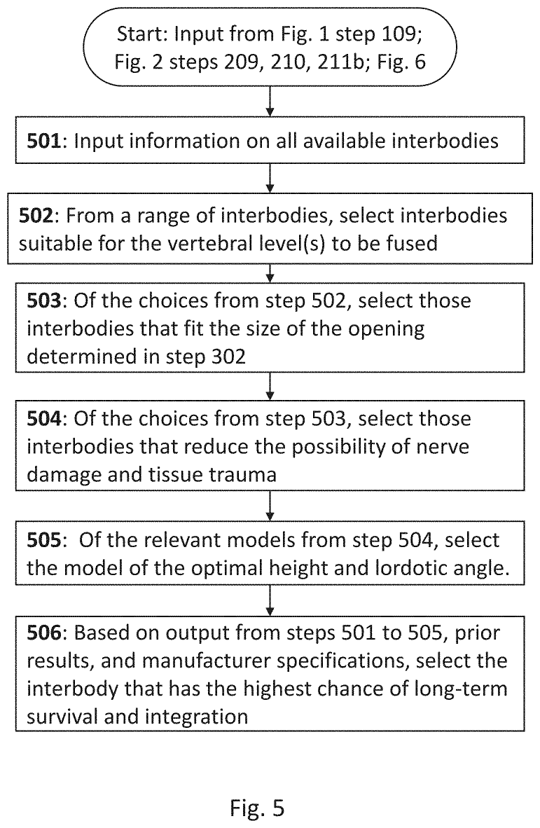

Reference is now made to FIG. 5, showing an exemplary decision-making tree for selection of the optimal interbody for lumbar fusion in a given individual, taking into consideration the information and decisions made in the methods described in the previous figures. Factors to be considered in the choice of implant comprise at least some of: selecting the interbody that 1) best corrects the patient's intervertebral disc defect, 2) has the best chance of long-term survival and integration, 3) allows for insertion that reduces the possibility of nerve damage and tissue trauma, 4) minimizes bone removal, and 5) is cost-effective. In a given subject, the order of importance of various factors may be different from the important factors for another subject; thus, the order of steps in FIG. 5 may be customized for each patient, such that the most critical factor(s) are considered first. The decision-making process for point 2) above may be aided by the methods disclosed in International Published Patent Application WO 2018/131045 for “Method and Apparatus for Image-based Prediction of Post-operative Spinal Pathologies”, having a common assignee with the present application.

The size and shape of manufactured spinal interbody implants range widely, depending on the manufacturer and style (step 501). For a given vertebral level and patient characteristics, the ideal interbody device is one that is rigid enough to maintain stability, but with a similar elastic modulus of bone to prevent subsidence and stress-shielding and having good osteoconductive properties (step 502). Additional factors to consider are the size and shape of the interbody relative to the height of the vertebral disc it is meant to replace, as a vertebral implant having greater dimensions would require a larger opening to enable insertion (step 503). Also considered in the selection of a specific interbody is information provided by the manufacturer regarding the full range of indications and contraindications for the device. The interbody cage options that reduce the possibility of nerve damage and tissue trauma for a given patient should be selected (step 504). The selected interbody cage should be of the correct height and anterior-posterior lordotic angle for the intervertebral disc it is replacing (step 505). Any other number of variables can be included in the paradigm for selecting the best interbody, depending on which factors the surgeon or the system determines are most important to optimize; those that are irrelevant for a given patient may be disregarded in the selection process.

A combination of factors may be considered in selection of the best interbody: based on the height of the disc to be replaced, the lordotic angle, and spinal level (step 605); based on the surgical approach (FIGS. 1 and 2) and the amount of bone that could safely be removed in this patient (FIGS. 3, 4, and 5) depending on underlying bone-related disease (arthritis, facet joint instability, osteoporosis, etc.)

Once the surgical path has been planned for a selected interbody and a given surgical approach, as described in FIG. 3 and in the co-pending US Provisional Application “Surgical Path Planning using Artificial Intelligence for Feature Detection”, the system assesses if the predicted outcome from this path would provide satisfactory results. In this context, a satisfactory result would comprise at least some of: 1) the ability to provide adequate access to the surgical site, 2) acceptable time of dissection to reach the site, and 3) ability of the patient to tolerate the requirements of the surgical procedure. At this point, the system then either decides that this approach is not ideal and returns to consider other approaches, or proceeds with creating a preoperative plan for execution by the surgical robotic system under the instructions of the controller.

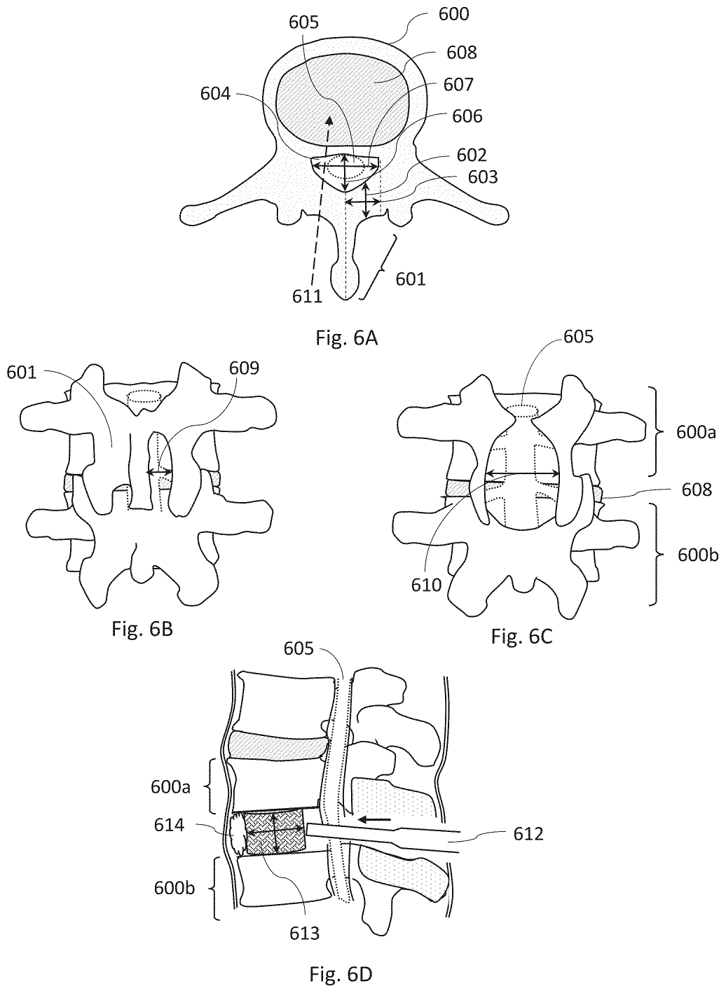

Reference is now made to FIGS. 6A to 6D, showing an exemplary implementation of the methods to plan a posterior approach to either a single level spinal fusion or an AIDR. Based on the preoperative images, the system measures each of the distances shown in FIG. 6A, to acquire a three-dimensional map of the relevant vertebrae, comprising barriers and potential spaces. In FIG. 6A, a lumbar vertebra 600 as viewed from above. The vertebral lamina 501 forms an arched roof over the spinal canal 604 and provides protection to the neural tissues enclosed in the thecal sac 605, as well as a bony barrier preventing access to the intervertebral disc space 608. A lumbar vertebral lamina is typical 11-16 mm in width, as shown by the double-headed arrow 603; and 16-22 mm in thickness, as shown by the double-headed arrow 602. The lumbar spinal canal 604 in a typical adult ranges from roughly 12-29 mm in the anterior-posterior dimension, as shown by the double-headed arrow 606; and 19-43 mm from side to side, as shown by the double-headed arrow 607. These dimensions are averages found in normal adult individuals; in patients with spinal stenosis and other medical conditions necessitating surgical decompression, the distances may be less. Dashed arrow 611 indicates the approach to the intervertebral space taken during a PLIF procedure. The relevance of the size of the spinal canal and the vertebral lamina becomes clear in the following FIGS. 6B, 6C, and 6D, as a surgical opening in the lamina is planned to enable insertion of the intervertebral prosthesis or artificial disc.

In FIGS. 6B and 6C, the spinal column is viewed from the posterior aspect, showing two adjacent lumbar vertebrae, 600a and 600b, with an intervening vertebral disc 508. For ease of viewing, identical features are numbered only in FIG. 5C; the same numbering applies to FIG. 5B. The disc 608 will be replaced with a prosthetic interbody. To enable access to the intervertebral space, a laminotomy, hemilaminectomy, (FIG. 5B) or full laminectomy (FIG. 5C) is performed to remove the overlying vertebral lamina. In a hemilaminectomy, the amount of bone removed, and thus potential destabilization of the spine, is reduced, resulting in a narrower opening 609 than occurs with a full laminectomy 610. In any case, the opening must be large enough to allow both gentle retraction of the neural tissue 605, and insertion of the interbody. Mean intervertebral disc height in the lower lumbar segments is in the range of 11 mm. Whereas the volume of a replacement interbody varies greatly, examples of a type of interbody implant currently available are in the range of 8.5 mm wide×22-28 mm long×6-17 mm high. These dimensions require an opening in the vertebral lamina at least as large as the two shorter outer dimensions of the interbody to be inserted. As the interbody may be greater in at least one dimension than the opening, in some implementations of the disclosed methods, a path-finding method is used to optimize the size of the opening and the orientation in which the interbody is to be inserted and maneuvered within the patient's anatomy. The system takes into account the findings from FIG. 4 steps 403 and 404, such that patients having reduced vertebral body strength, concurrent inflammatory disease, or abnormal motion of the vertebral pedicles, have lower allowable limits for the amount of bone that may be removed.

In FIG. 6D, the spinal column is shown in a lateral/side view, with anterior to the left and posterior to the right of the drawing. The thecal sac enclosing neural tissue 605 has been retracted laterally, as is shown by its position behind the tool 612, and a prosthetic interbody 614 and a bone graft 613, expected to grow into the hollow interbody, have been inserted between the two vertebrae 600a and 600b by a surgical tool 612. The interbody 614 is shown schematically in one dimension; other configurations of interbody extending over a larger region of the intervertebral disc space with a hollow center, or two smaller interbodies, may be used. Based on the three-dimensional relationships among the vertebrae, size and shape of the laminectomy opening, and the three-dimensional volume of the interbody, the system must calculate the minimal amount of laminar bone to remove to ensure the safe and successful insertion of the interbody. In some implementations of the disclosed methods, the calculation is accomplished by simulating force prediction on the delaminated vertebra in the 3D preoperative images with finite element analysis. The analysis may also use elements shown in WO2018/131044 for “Dynamic Motion Global Balance”, having a common assignee as the present application, and other applications mentioned herewithin above. It is to be understood that the bone graft and hollow interbody are illustrating one exemplary use of the method, which is applicable to any number of specific surgical implementations and approaches. In the case of an AIDR, an AID would be inserted in place of the interbody, and no bone graft would be used.

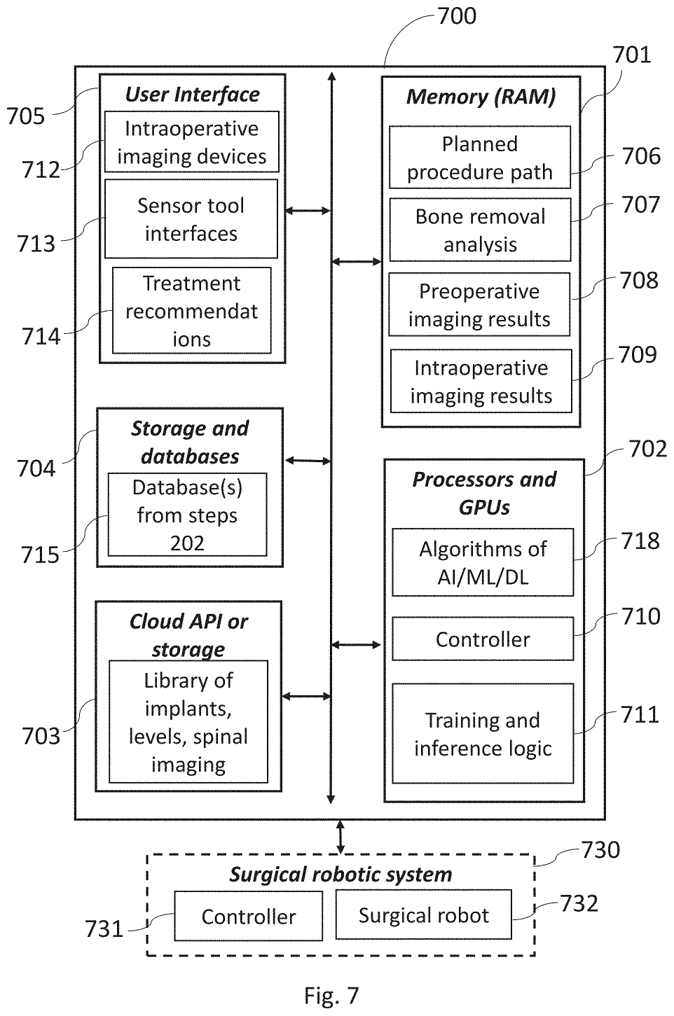

Reference is now made to FIG. 7, schematically showing the components of an exemplary system 700, for implementation of some of the methods described in the present disclosure. An exemplary implementation of the system comprises a processor 702, a memory 701, a user interface 705, at least one database or channel 704 comprising clinical information regarding past cases of low back pain (from at least step 202), and optionally a cloud API or storage 703 comprising a library of implants and optionally, spinal imaging results, ideal implants for specific spinal levels, and manufacturers' data on prosthetic hardware implants. The memory component 701 comprises both sources of input for the processing of the method, such as the preoperative CT or MM images 708, as well as storing the output of the method, i.e., planned procedure path 706, intraoperative imaging results 709, and the bone removal analysis 707, output from FIG. 3. The processor comprises artificial intelligence algorithms 718, a controller 710, and training and inference logic 711. The system 700 is configured to communicate with and provide instructions to a robotic surgical system 730, comprised of a controller 731 and surgical robot 732, which carry out the operation according to the system output according to the planned procedure path 706. The processor 702 integrates all of the various inputs and generates an output comprising a surgical path plan or instructions 706, which are provided to the robotic controller 731, to carry out surgical access to the target site. Further components of the system 700 comprise the user interface 705, through which the surgeon or other health care provider interacts with the system.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

The apparatuses and methods described in this disclosure may be partially or fully implemented by one or more computer programs executed by one or more processors. The computer programs include processor-executable instructions that are stored on at least one non-transitory tangible computer readable medium. The computer programs may also include and/or rely on stored data.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure.

What is claimed is:

1. A system of planning surgical access to an intervertebral disc space of a subject for robotic insertion of prosthetic hardware, thereby to create a bone-hardware assembly, the system comprising:
at least one processor executing instructions stored on at least one non-transitory storage medium, to cause the at least one processor to:
i) analyze collected clinical data on the subject to detect conditions that may affect either spine strength or the expected useful lifetime of the bone-hardware assembly;
ii) use a path-finding algorithm to plan a path for the surgical access using a virtual representation of the prosthetic hardware superimposed on a three-dimensional preoperative image set of the region of the intervertebral disc space of the subject; and
iii) use any of the detected conditions and the planned path to determine a minimal amount of vertebral bone to be removed to allow (a) removal of the intervertebral disc and (b) robotic insertion of the prosthetic hardware along the planned path,
such that an increase is achieved in at least one of the likelihood of a favorable clinical outcome or the expected lifetime of the bone-hardware assembly.

2. The system according to claim 1, wherein the minimal amount of vertebral bone to be removed is determined using a path-finding algorithm.

3. The system according to claim 1, wherein the three-dimensional preoperative image set is one of MRI, CT, or reconstructed two-dimensional X-ray images.

4. The system according to claim 1, wherein a favorable surgical outcome is defined by at least two of a) long-term survival of the bone-hardware assembly, b) resolution of the subject's pain or loss of function, and c) lack of secondary complications from the surgical procedure resulting in injury to structures susceptible to damage.

5. The system according to claim 1, wherein minimizing a risk of failure of the bone-hardware assembly takes into consideration calculation and optimization of spinal alignment parameters.

6. The system according to claim 1, wherein the type of prosthetic hardware comprises an interbody cage, and wherein the processor is further configured to determine how much force is necessary to safely insert the interbody between the vertebrae.

7. The system according to claim 1, wherein the type of prosthetic hardware comprises an interbody cage, and wherein the processor is further configured to generate instructions to cause a surgical robotic system to perform robotic insertion of the interbody after providing surgical access to the intervertebral disc space.

8. The system according to claim 1, wherein planning the minimal amount of vertebral bone to be removed takes into account protection of the vertebral end plates and avoidance of structures susceptible to damage.

9. The system according to claim 1, wherein the at least one processor further uses training and inference logic to at least one of:
i) analyze the collected clinical data on the subject;
(ii) use a path-finding algorithm to plan the path for the surgical access; or
(iii) use any of the detected conditions and the planned path to determine a minimal amount of vertebral bone to be removed,
such that a greater increase is achieved in at least one of the likelihood of a favorable clinical outcome, or the expected lifetime of the bone-hardware assembly.

10. The system according to claim 1, further comprising a surgical robot having a controller configured to receive input from the processor, such that the surgical robot carries out the planned surgical access.

11. The system according to claim 1, wherein the bone-hardware assembly comprises (i) an inserted prosthetic intervertebral disc and its adjacent vertebral bodies; or (ii) at least one interbody and associated hardware needed for a spinal interbody fusion.

12. A system for determining the suitability of a subject with spinal pain, for a surgical procedure to decompress or replace an intervertebral disc, the system comprising:
at least one processor executing instructions stored on at least one non-transitory storage medium, to cause the at least one processor to:
i) analyze a database of medical history information of a reference population comprising patients having previously undergone surgical procedures for spinal pain, to categorize outcomes of the surgical procedures according to clinical and demographic parameters;
ii) use the analyzed database to classify the subject based on the clinical and demographic parameters of the subject; and
iii) based on the classification of the subject, determine at least one of:
a) the suitability of the subject for surgical treatment;
b) the type of surgical procedure to perform on the subject; or
c) the surgical approach to perform the surgical procedure;
wherein the determination results in optimization of the expected outcome of the surgical procedure on the subject.

13. The system according to claim 12, wherein categorizing outcomes of surgical procedures comprises ranking the degree of spinal pain and the physical disability of patients preoperatively and post-operatively according to a numerical scale.

14. The system according to claim 13, wherein the numerical scale is either of the Neck Disability Index (NDI) or the Oswestry Disability Index (ODI).

15. The system according to claim 12, wherein the determination of suitability for surgical treatment is based on consideration of at least one of clinical and demographic factors, underlying bone disease, or preexisting conditions.

16. A system for planning the selection of an artificial prosthesis to replace an intervertebral disc, comprising:
a) a memory configured to store a selected surgical procedure and a surgical approach for performing insertion of an artificial disc prosthesis on a subject,
b) a channel providing access to information on at least some of dimensions, shape, indicated surgical use, indicated vertebral levels, material composition, and success rate of available artificial disc prostheses, and
c) a controller accessing artificial intelligence algorithms, to i) analyze the information on the available artificial disc prostheses, and ii) select an artificial disc prosthesis for the subject, such that the long term outcome of the surgical procedure on the subject is optimized.

17. The system according claim 16, wherein at least one of the algorithms takes into account the optimal height and lordotic angle of the intervertebral disc to be replaced by the artificial prosthesis.

18. The system according to claim 16, wherein optimizing the expected outcome of the surgical procedure is defined by at least two of a) long-term survival of the artificial prosthesis, b) resolution of the subject's disability, and c) lack of secondary complications from the surgical procedure.

19. The system according to claim 16, further comprising training data on outcomes of previous surgical procedures and surgical approaches using the available artificial disc prostheses.

20. The system according to claim 19, wherein the training data is used by the algorithms to predict at least one of a) long-term survival of the artificial prosthesis, b) resolution of the subject's disability, and c) lack of secondary complications from the surgical procedure.
